(19) United States
(12) Patent Application Publication
(10) Pub. No.: US 2013/0029312 A1
(43) Pub. Date: Jan. 31, 2013

(54) INTEGRAL SPERM PREPARATION FOR INTRACYTOPLASMIC SPERM INJECTION

(71) Applicant: James B. Johnston, Ambler, PA (US)

(72) Inventor: James B. Johnston, Ambler, PA (US)

(73) Assignee: BIOCOAT, INC., Horsham, PA (US)

(21) Appl. No.: 13/192,002

(22) Filed: Jul. 27, 2011

(51) Int. Cl. C12N 5/076; C12M 3/00

(52) U.S. Cl. 435/2; 435/283.1

(57) ABSTRACT

Methods, systems and apparatus for preparing sperm for ICSI by combining selective enrichment benefits of sperm gradient preparation, sperm swim-up preparation and hyaluronan-binding for selection of mature sperm into a single processing unit. The single processing unit of the invention reduces operator labor steps and time, is efficient, easy to use, offers quick processing time and provides highly motile, mature and specific individual sperm that retain desired sperm characteristics selected by each individual selection principal that are ready for capture and use in ICSI.

### BACKGROUND OF THE INVENTION

1. Field of the Invention

The present invention relates to methods, systems and apparatus for preparing spermatozoa for Intracytoplasmic Sperm Injection.

2. Description of Related Art

Intracytoplasmic sperm injection (ICSI) is an in vitro fertilization (IVF) procedure in which a single sperm is injected directly into a mature egg (oocyte). Generally, several oocytes are extracted from a female and, under a microscope, one of the oocytes is held with a specialized pipette that stabilizes the oocyte with gentle suction applied by a microinjector. A thin, sharp, and hollow micropipette is used to immobilize and pick up a single sperm, followed by carefully inserting the micropipette through the shell (oolemma) of the oocyte and into the inner part (cytoplasm) of the oocyte. The sperm is then released into the cytoplasm, and the micropipette is carefully removed. After the procedure, the oocyte is placed into a cell culture and examined the following day for signs of normal fertilization. Once fertilization is successful, the embryo is transferred to the female's uterus.

ICSI has become the dominant treatment for infertile couples wherein the infertility involves a male factor. Again, in the ICSI process an individual, isolated sperm is injected directly into the oocyte to create a conception. Since the individual selected sperm makes direct parental contribution to the conception, it is important that it be of the highest possible quality. Therefore, prior to selection of the sperm, the semen sample is processed to remove contaminants (bacteria, non-sperm cells, inhibitory factors in the seminal plasma, dead sperm and cellular debris) and to enrich for normal, motile, mature sperm.

At the inception of in vitro fertilization, sperm processing consisted of a simple washing by repeated dilution, centrifugation and re-suspension in fresh clean media. This treatment removed inhibitory factors in the seminal plasma and allowed the sperm to undergo capacitation to become fertile.

Over the years the swim-up technique was introduced which involves motile sperm swimming away from contaminants, while non-motile sperm remains behind. In this approach, raw semen is first centrifuged and re-suspended in a clean, artificially constructed support medium such as modified Human Tubal Fluid (mHTF) or other sperm-compatible medium. Sedimentation and re-suspension of the sperm removes the majority of the cellular debris and soluble inhibitory factors toxic to sperm but retains dead cells, some bacteria and contaminating non-sperm cells. To perform the swim-up, re-suspended sperm are re-concentrated by centrifugation, retaining a small volume of overlying support medium, and incubated for 30 to 60 minutes at 37° C. without any agitation. During the incubation period, motile sperm in the pellet swim up into the overlying fluid. Removal of part of the overlying fluid captures these sperm and separates them from the pellet, which retains the dead sperm and non-motile contaminating cells.

While swim-up techniques involving several centrifugation steps are widely used, such methods have several disadvantages. For instance, these known methods recover only a small proportion of the initial sperm in the semen sample. Such swim-up techniques are also labor intensive and tedious requiring multiple processing steps over several hours. In particular, swim-up with centrifugation steps require 1 or more hours of operator time, plus 30 to 60 minutes of swim-up incubation time. Another disadvantage of these known swim-up techniques is that while such techniques provide highly motile sperm, these highly motile sperm often include underdeveloped motile sperm and physiologically immature sperm, in addition to motile contaminating bacteria being present within the captured sample from the processed overlying fluid.

Another advancement in the in vitro fertilization field is the discontinuous density gradient centrifugation processing. Sperm density increases late in the process of sperm development. In known discontinuous density gradient centrifugation techniques the denser sperm are separated from their progenitors by centrifugation on the discontinuous density gradient. Commercial density gradient sperm isolation kits that utilize this technique to prepare sperm for ICSI include Pure Sperm® 40/80 and ISolate®. Yet, while the centrifugation accelerates the sinking of the denser sperm, it also detrimentally causes both motile and non-motile sperm, dead sperm and motile, fully formed but physiologically deficient sperm to sink as well. The centrifugation processes are also labor intensive and tedious requiring multiple processing steps performed by an operator over an extended time.

ICSI methods based on specific sperm binding reactions have also been developed. For instance, specific binding of sperm to annexin V has been used to remove sperm undergoing apoptosis (programmed cell death), thereby improving the quality of the resulting sperm population.

Another more notable binding technique is the binding of viable, mature sperm to hyaluronan (HA), which is the main constituent of the gel layer (cumulus oophorus) surrounding the oocyte. It is believed that sperm binding to this portion of the oocyte is an essential step in the normal process of conception. HA binding methods allow the selection and capture of individual sperm displaying a desired trait.

A known HA sperm selection device is the PICSI® Sperm Selection Device from Biocoat, Inc., which provides microscopic droplets of HA hydrogel attached to a culture dish. An ICSI operator then provides prepared sperm directly onto these microscopic droplets, whereby physiologically mature sperm will bind thereto while immature sperm will not. The ICSI operator selects from among the bound sperm, recovers individual bound sperm, and uses the recovered sperm in further ICSI processing. While HA sperm binding is a relatively simple and fast procedure to isolate physiologically mature sperm, the physiologically mature sperm that may be bound to the microscopic droplets of HA hydrogel may be contaminated by unbound, undesirable sperm and other contaminants, depending upon the sperm preparation used to inoculate such HA microscopic droplets.

While several ICSI processing techniques currently exist, none yield highly motile, mature and specific individual sperm that retain desired characteristics selected by each individual selection principal in a simple, directly accessible form that are ready for selection and capture and use in ICSI.

In particular, both the swim-up and discontinuous density gradient techniques require numerous processing steps and require the use of centrifugal force, which may damage the sperm. Also, each of these techniques yield a population of sperm enriched for desirable characteristics, but do not necessarily identify and recover specific individual sperm possessing one or more desired properties.

Further, the yields of sperm populations may include highly motile sperm (e.g., by swim-up), however, such highly motile sperm may include underdeveloped or physiologically highly motile immature sperm as well as highly motile bacteria. In sperm populations obtained by discontinuous density gradient centrifugation, such populations may undesirably include both motile and non-motile sperm, dead sperm and motile, fully formed but physiologically deficient sperm. Sperm recovered by HA sperm selection may be specific individual sperm, however, these sperm may undesirably be contaminated by unbound, undesirable sperm and other contaminants from the sperm preparation.

Other ICSI techniques have been implemented to address the undesirable attributes of the above ICSI techniques by sequentially combining two of such techniques, one after the other, to improve the quality of the processed sperm. For instance, Chen, S. U. et al., “Combination of direct swim-up technique and discontinuous Percoll gradient centrifugation for sperm preparation of oligoasthenozoospermic samples” Arch. Androl. 37 (2):103-9 (1996), discloses the use of swim-up and density gradient separation, however, the sperm were processed by these two methods one after the other, markedly increasing the steps, labor intensiveness, time and effort required for processing. These sequential ICSI technique processes are more time consuming and labor intensive than the individual methods alone, and none yield sperm in a simple, directly accessible form, ready for selection and capture and use in ICSI.

Accordingly, a need continues to exist in the art for improved ICSI methods, systems and apparatus for yielding highly motile, mature and specific individual sperm that retain desired characteristics, selected by individual selection principals, in a simple, directly accessible form whereby such sperm are ready for selection and capture and use in ICSI.

### SUMMARY OF THE INVENTION

Bearing in mind the problems and deficiencies of the prior art, it is therefore an object of the present invention to provide methods, systems and apparatus that improve sperm quality for ICSI.

It is another object of the present invention to provide methods, systems and apparatus that yield highly motile, mature and specific individual sperm that retain desired characteristics in a simple, directly accessible form.

A further object of the invention is to provide methods, systems and apparatus that easily, efficiently and with reduced operator labor provide sperm with improved qualities for selection and capture and use in ICSI.

Another object of the invention is to provide methods, systems and apparatus that eliminate the need for centrifugation in ICSI sperm processing and isolation to avoid centrifugal-mechanical damage to the resultant sperm.

Still other objects and advantages of the invention will in part be obvious and will in part be apparent from the specification.

The above and other objects, which will be apparent to those skilled in the art, are achieved in the present invention which is directed to a method for preparing sperm for Intracytoplasmic Sperm Injection (ICSI) by providing a processing unit that includes a hollow vertical unit attached to a horizontal support structure. The hollow vertical unit has a first end adjacent the support structure to provide a leak-proof seal and a second end having an opening. A hyaluronan layer resides on the horizontal support structure in a location corresponding to the first end of the hollow vertical unit, such that, a portion of the hyaluronan layer is exposed within the hollow vertical unit.

A density gradient media layer is deposited into the opening of the hollow vertical unit to entirely cover the exposed hyaluronan layer within the hollow vertical unit. A semen layer is then deposited directly over the density gradient media layer to form a defined horizontal density gradient interface between the density gradient media layer and the semen layer. The density gradient media layer has a density greater than a density of the semen layer and lower than a density of fully-formed motile sperm.

The processing unit is allowed to stand undisturbed for an amount of time to allow fully-formed motile sperm swim in a downward vertical direction from the semen layer, through the defined horizontal density gradient interface, through the density gradient media layer and toward the hyaluronan layer to bind to the hyaluronan layer. The semen and density gradient media layers are then removed, and the hyaluronan layer having the fully-formed motile sperm bound thereto is accessed by removing the hollow vertical unit from the horizontal support structure.

In one or more embodiments, the support structure may be a transparent material for viewing the hyaluronan layer to observe binding of the fully-formed motile sperm to the hyaluronan layer. The density gradient media layer may have a density ranging from about 1.050 g/mL to about 1.150 g/mL, and may have a pH ranging from about 7.2 to about 7.6. The density gradient media layer may be selected from PVP-coated silica media, silane-coated silica media or iodixanol media.

In other embodiments, the semen sample may be neat semen, diluted raw semen, freshly thawed frozen semen, treated semen, compromised semen, or even combinations thereof. The fully-formed motile sperm binds to the hyaluronan layer without the need for centrifugal forces. In doing so, the processing unit may be allowed to stand for an amount of time ranging from about 5 minutes to about 30 minutes. Due to the defined horizontal density gradient interface and the density gradient media layer, the following are prevented from contacting and binding to the hyaluronan layer: motile underdeveloped motile sperm, motile physiologically immature motile sperm, motile contaminants, non-motile sperm, under-developed sperm, dead sperm, dead cells, bacteria, cellular debris, non-sperm cells, seminal plasma, non-motile contaminating cells, soluble inhibitory factors, and combinations thereof.

In still other embodiments the method may further include rinsing the hyaluronan layer having the fully-formed motile sperm bound thereto prior to ICSI sperm selection. The method may further include providing the support structure having the hyaluronan layer with the fully-formed motile sperm bound thereto within an ICSI culture dish for ICSI sperm selection. Alternatively, the support structure may be an ICSI culture dish for ICSI sperm selection.

The invention is also directed to an apparatus for preparing sperm for Intracytoplasmic Sperm Injection that includes a support structure having a horizontal plane, a hyaluronan layer on the support structure, and a hollow vertical unit in communication with the support structure. The hollow vertical unit has a length that extends vertically, with respect to the horizontal plane of the support structure, and has a first end adjacent the support structure to provide a leak-proof seal and a second end space apart from the support structure. The second end has an opening for entry of a fluid column into the hollow vertical unit. The hyaluronan layer resides at the first end of the hollow vertical unit and has a surface area of hyaluronan layer exposed within the hollow vertical unit.

In certain embodiments, the support structure may be of a transparent material for viewing the hyaluronan layer. The hyaluronan layer may reside across the support structure, such that, the hollow vertical unit contacts the hyaluronan layer on the support structure to form the leak-proof seal between the hollow vertical unit and the hyaluronan layer. Alternatively, the hyaluronan layer may be a region of hyaluronan residing at a specific location on the support structure, whereby the hollow vertical unit contacts the support structure to form the leak-proof seal between the hollow vertical unit and the support structure. As such, the region of hyaluronan resides entirely within the hollow vertical unit, and the hollow vertical unit encases the region of hyaluronan.

The hollow vertical unit may be removably attached to the support structure. It may be securely connected to the support structure using a breakable connection. This breakable connection may be made by a breakable chemical connection or a breakable thermally-bonded connection, whereby a user may physically break the connection to remove the hollow vertical unit from the support structure and gain access to the hyaluronan layer.

In still other embodiments, the invention is also directed to a system for preparing sperm for Intracytoplasmic Sperm Injection that includes a support structure having a horizontal plane, a hyaluronan layer on the support structure, and a hollow vertical unit in communication with the support structure. The hollow vertical unit has a length that extends vertically, with respect to the horizontal plane of the support structure, and has a first end adjacent the support structure to provide a leak-proof seal and a second end space apart from the support structure. The second end has an opening for entry of a fluid column into the hollow vertical unit. The hyaluronan layer resides at the first end of the hollow vertical unit and has a surface area of hyaluronan layer exposed within the hollow vertical unit.

The system may further include a density gradient media layer within the hollow vertical unit to entirely cover the exposed hyaluronan layer therein, and a semen layer directly over the density gradient media layer to form a defined horizontal density gradient interface therebetween. The density gradient media layer has a density greater than a density of the semen layer and lower than a density of fully-formed motile sperm, whereby after standing undisturbed for an amount of time, fully-formed motile sperm swim vertically downward from the semen layer, through the defined horizontal density gradient interface, through the density gradient media layer and toward the hyaluronan layer to bind thereto, while non-motile matter, motile underdeveloped motile sperm, motile physiologically immature motile sperm, and motile contaminants are prevented from contacting the hyaluronan layer by the defined horizontal density gradient interface and the density gradient media layer.

### BRIEF DESCRIPTION OF THE DRAWINGS

The features of the invention believed to be novel and the elements characteristic of the invention are set forth with particularity in the appended claims. The figures are for illustration purposes only and are not drawn to scale. The invention itself, however, both as to organization and method of operation, may best be understood by reference to the detailed description which follows taken in conjunction with the accompanying drawings in which:

FIG. 1 is a side view of an apparatus in accordance with one or more embodiments of the invention for preparing sperm for ICSI.

FIGS. 2A-2G are side perspective views showing an apparatus and method in accordance with one or more embodiments of the invention for preparing sperm for ICSI.

### DESCRIPTION OF THE PREFERRED EMBODIMENT(S)

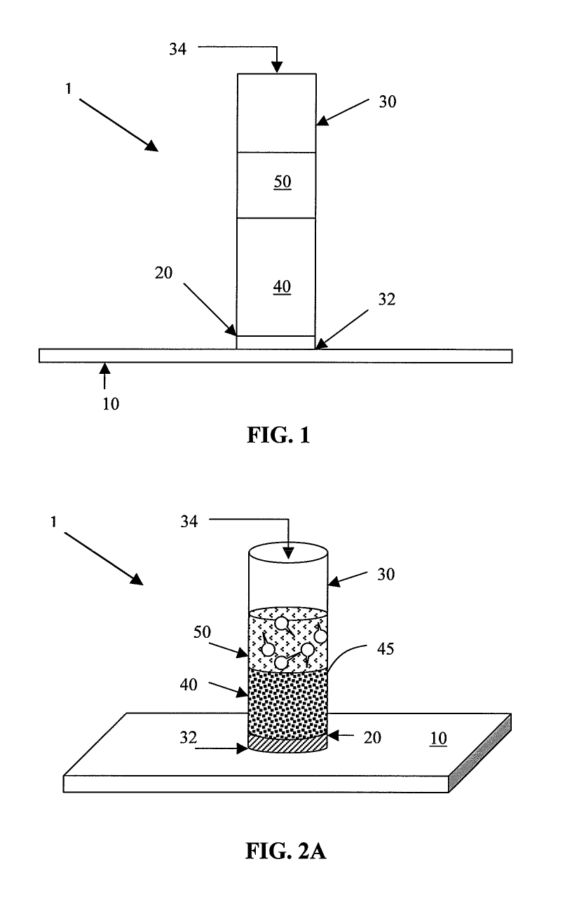

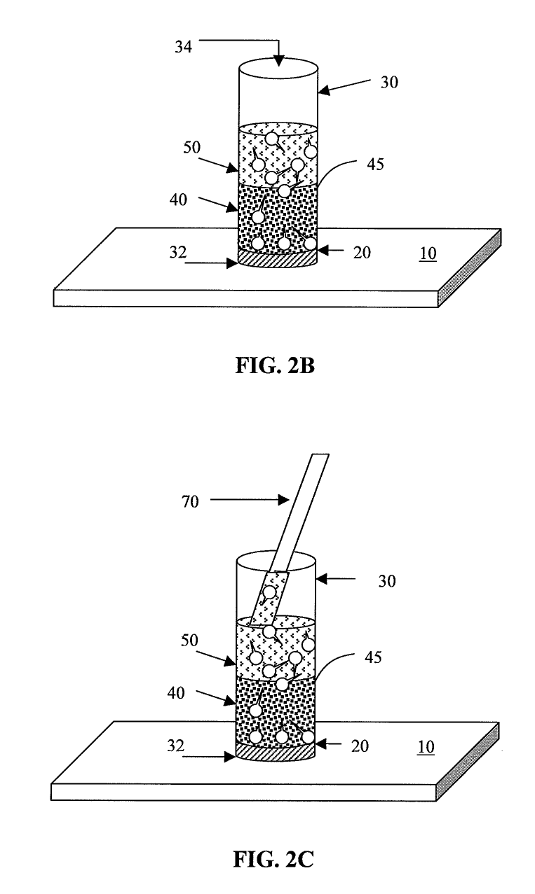

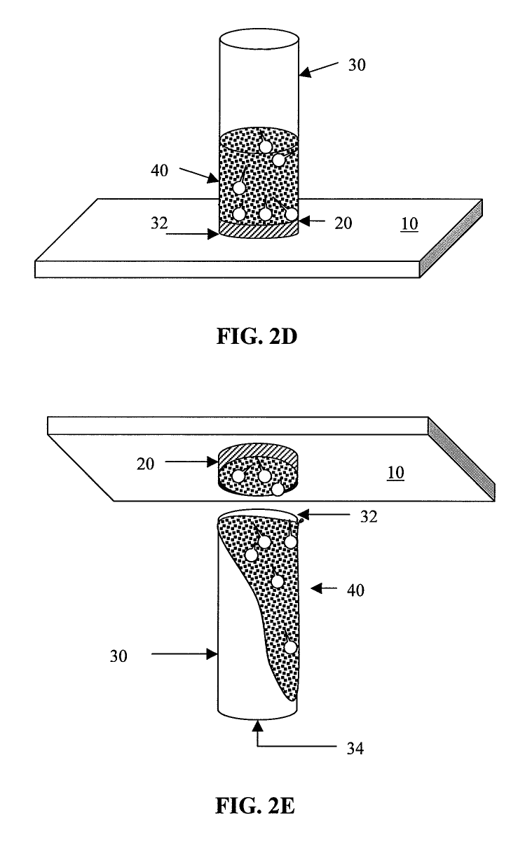

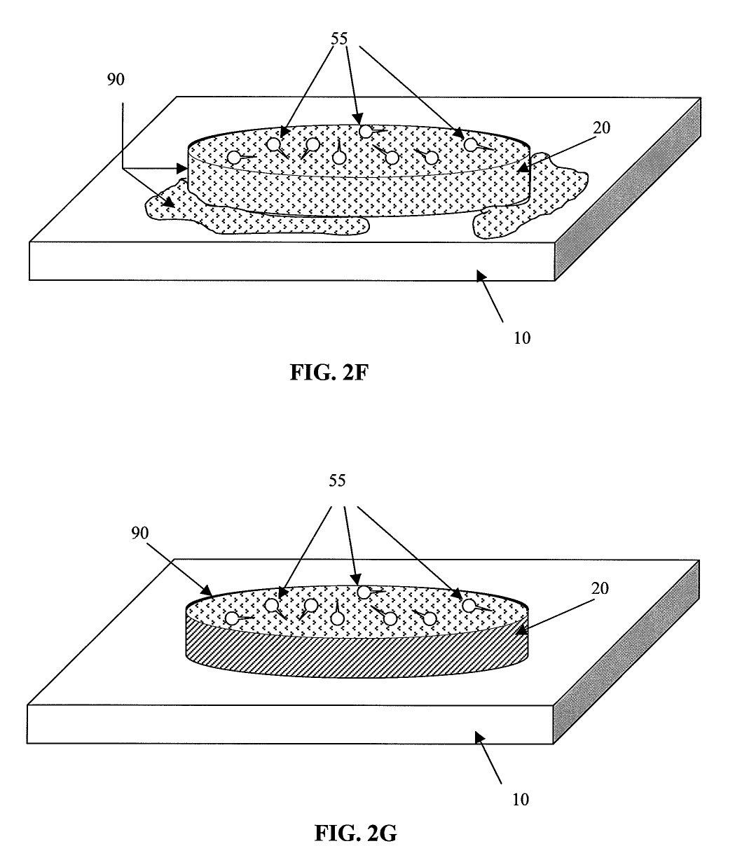

In describing the preferred embodiment of the present invention, reference will be made herein to FIGS. 1-2G of the drawings in which like numerals refer to like features of the invention.

In accordance with the various embodiments, the invention is directed to methods, systems and apparatus that improve sperm quality for ICSI by processing sperm using several different ICSI techniques within a single device. In particular, the various embodiments of the invention combine the concept of ICSI sperm swim-up selection, density gradient selection and HA-binding selection using principals unique to the invention within a vertically oriented device of the invention. The invention reduces operator labor steps and time (as compared to each above-discussed known ICSI process), and provides highly motile, mature and specific individual sperm that retain desired characteristics selected by each individual selection principal that are ready for capture and use in ICSI.

Referring now to the drawings, FIG. 1 shows a cross sectional side view of an ICSI processing unit 1 in accordance with various embodiments of the invention. The ICSI processing unit 1 includes a thin, flat, optically transparent support structure 10 having one or more regions of hyaluronan (HA) located thereon. The HA layer may be chemically bound or attached to the transparent support structure 10. The optically transparent support structure 10 may be composed of glass or a plastic material, whereby the support structure may be placed on an inverted microscope for magnification through the bottom of the support structure using the microscope to view the upper surface of such support structure, and in particular, to view the HA region(s). In one or more embodiments, the HA region may be an HBA® Sperm-Hyaluronan Binding Assay slide from Biocoat, Inc.

As shown in FIG. 2A, attached to the support structure 10 is a hollow vertical unit 30 that may have openings at both ends thereof. The top end of the vertical unit may have an opening 34 for allowing media and sperm specimens to be provided inside the vertical unit 30. The bottom end 32 of the vertical unit 30 may be adjacent the transparent support structure 10 and may contain within this bottom end a region of HA as an HA layer 20. That is, the hyaluronan may entirely coat a surface area of the support structure 10, or specific regions of hyaluronan may be provided at multiple locations across the surface area of the support structure 10. The vertical unit 30 may contact portions of the hyaluronan layer, contact portions of the regions of hyaluronan, or it may directly contact with underlying support structure 10. In each embodiment, the vertical unit 30 encases a portion, or all, of the hyaluronan to provide HA layer 20 residing at the bottom of the vertical unit 30. In one or more embodiments, the HA layer 20 is attached to the support structure 10 and may reside entirely within the vertical unit 30. In these embodiments, the hollow vertical unit 30 has a diameter greater than the diameter of the HA layer 20, such that, the vertical unit 30 completely encompasses and encases the HA layer 20 at its bottom end 32.

The bottom end 32 of the vertical unit 30 may be in communication with the support structure 10, whereby the unit is preferably removably attached to the support structure 10, while also providing a leak-proof seal therewith. For instance, in one or more embodiments, a vertical unit 30 of 3 cm in length (height) may be attached to the support structure 10 to provide a leak-proof seal, such that, aqueous fluid provided therein has a leak-proof seal that may last for at least 2 hours, and preferably more.

The vertical unit 30 may be removably affixed to the support structure 10, or it may be separate from the support structure where it is simply placed or positioned on top of the support structure while still forming a leak-proof seal for ICSI processing in accordance with the invention. In those embodiments where the vertical unit 30 is affixed to the support structure 10, the vertical unit 30 may be chemically adhered to the support (e.g., through glue, adhesive, etc.). Alternatively, the vertical unit or tube may be thermally adhered to the support structure by heating the vertical unit/tube and contacting it to the support base resulting in attachment to the base by local melting of the support structure at and around the points of contact with the heated vertical unit or tube. It should be appreciated that while the vertical unit 30 is shown cylindrical in shape (i.e., as a vertical tube), the vertical unit may have any shape that is suitable for vertical ICSI processing in accordance with the invention.

A first media is provided within the vertical unit 30 to form a first media layer 40. This first media layer 40 is an aqueous fluid of known density that is greater in density than the density of a semen layer 50 provided on top of such first media layer 40. The media layer 40 is preferably non-toxic to sperm, and may have a pH ranging from about 7.2 to about 7.6, more preferably from about 7.3 to about 7.5, and most preferably from about 7.35 to about 7.45. Optionally the aqueous density medium may also include ions, nutrients, energy sources or other components that stabilize and protect sperm, for instance, such as those included in commercial density media such as NidaCon Pure® Sperm 80. Optionally, the aqueous density medium may include at least 0.50 mg/mL of albumin protein, preferably about 5.00 mg/mL, but no more than 30 mg/mL, of albumin protein. The albumin protein may be of animal origin, preferably human serum albumin or most preferably recombinant (synthetic) human albumins.

In one or more embodiments, the density of the media layer 40 may range from about 1.050 g/mL to about 1.150 g/mL, more preferably from about 1.095-1.130 g/mL, and most preferably of about 1.100 g/mL. The dense media layer 40 is in direct contact with the HA layer 20, such that, the semen layer 50 is completely isolated from the HA layer 20 by such dense media layer 40. The dense media layer 40 is preferably a layer of density gradient separation media, such as, Pure Sperm® 80 of NidaCon. The dense media layer 40 is introduced into the vertical unit 30 so that it directly contacts and entirely covers HA layer 20 residing at the bottom of the unit 30.

While not meant to limit the invention, in one or more embodiments the volume of media layer 40 provided within the vertical unit 30 may be about 0.75-0.80 mL within a vertical unit having an internal diameter of about 0.8-1.0 cm, corresponding to a column height of about 6.5 mm to 7 mm. In other embodiments, depending upon the amount of media layer 40 provided within the vertical unit 30, the sperm may have to swim down traversing through the media layer 40 a vertical distance ranging from about 1 mm to about 30 mm, preferably from about 4 to about 8 mm, and most preferably about 5 to about 7 mm. As discussed in more detail below in relation to the examples, a desired concentration of sperm bound to the HA layer is dependent upon the concentration of sperm within the semen layer 50, the dimensions of the vertical unit and the volume of the density medium layer provided therein.

Once the dense media layer 40 resides within the vertical unit 30 the semen layer 50 is provided within the vertical unit 30 so that it is directly over and contacts the dense media layer 40. The semen layer 50 may comprise neat (“fresh”) semen, diluted raw semen, freshly thawed frozen semen, treated semen (e.g., semen treated with proteolytic enzymes for liquefication), and the like. Regardless of the type of semen layer 50, such semen layer 50 is not subjected to centrifugal forces prior to its utilization, thereby preventing damage to the sperm within the semen sample due to mechanical forces.

The semen layer 50 is gently provided within the vertical unit 30 over the dense media layer 40 so as to form a defined horizontal interface 45 between the two layers 40, 50. This horizontal interface 45 is generated as a result of the different densities between the semen layer 50 and the underlying dense media layer 40, whereby such interface 45 resides horizontally within the vertical unit 30. In one or more embodiments, it is preferred that the vertical unit 30, containing the semen layer 50, underlying dense media layer 40 and horizontal interface 45, remain within the vertical position without any movement, and in particular without centrifugation, so that the solutions therein stabilize and the horizontal interface 45 is maintained. In so doing, the orientation of the liquid column containing the dense medium and semen remains vertical relative to the horizontal plane of the HA layer 20.

With the semen layer 50, dense media layer 40 and horizontal interface 45 residing vertical relative to the horizontal plane of the HA layer 20, sperm have a defined vertical path to follow for traveling from the semen layer 50, through the dense media layer 40 and down to the HA layer 20. This vertical liquid column of the less dense semen layer 50 over the more dense media layer 40 enables the vertically oriented density selection step in accordance with various embodiments of the invention. Since the present two-phase liquid column resides vertically, and as such sperm swim through this vertical path, only fully-formed motile sperm are dense enough to traverse the horizontal interface 45 residing between the different density layers and swim vertically through the underlying media layer 40 toward the HA layer 20.

The vertically oriented density selection step of the invention separates sperm from the semen layer 50 based on the different density gradients within the vertical unit 30 and based on the density of the sperm. Sperm density is strongly affected by the extent of sperm development, increasing as the progenitor cells are formed into finished sperm. During development, the sperm's DNA counter ions, proteins called histones, are exchanged for much smaller and compact counter ions called protamines. The resulting re-packaged DNA is much denser than the histone-DNA complex. In addition, in the late stage of sperm development, the cytoplasm of the developing sperm is removed from the sperm by extrusion. Since cytoplasm is less dense than protamine-DNA, any retention of cytoplasm lowers the sperm density. Fully formed sperm lack cytoplasm, have very compact DNA, and consequently, are denser than underdeveloped sperm, contaminating cells, bacteria and sperm progenitor cells.

In accordance with one or more embodiments of the invention, sperm within the semen layer 50 are positioned over the discontinuous density gradient of the dense media layer 40. This media 40 has a density less than that of fully formed sperm within the semen layer 50, but higher than that of under-developed sperm, bacteria, cellular debris, non-sperm cells and seminal plasma, and the like, residing within the semen layer. As such, only the fully-formed motile sperm are dense enough to traverse the horizontal interface 45 residing between the different density layers and swim vertically through the underlying media layer 40 toward the HA layer. All other matter having a density less than that of the media layer 40 will float on top of such media layer 40, and not cross the horizontal interface 45.

The fully-formed motile sperm that are more dense than the media layer 40 are allowed to sink therein and swim vertically through the media 40 towards the HA layer 20, without the need for centrifugation or centrifugal forces. The critical density of the media 40 that allows fully formed human sperm to sink and swim vertically therethrough, but floats under developed sperm and contaminants, lies in the range of about 1.080 g/mL to about 1.100 g/mL. In one or more embodiments, the density gradient media suitable for use in accordance with the invention for providing sperm-compatible fluids of the required density include, but are not limited to, PVP-coated silica (e.g., Percoll polymer), silane-coated silica (e.g., Pure Sperm® 80 of NidaCon, or Isolate® of Irvine Scientific), iodixanol (e.g., Optiprep® of Sigma-Aldrich), and the like. However, it should be appreciated that any suitable sperm compatible or aqueous-based, sperm compatible media may be implemented as the dense media layer 40, such as, any aqueous based sperm compatible media having a density range from about 1.050 g/mL to about 1.150 g/mL.

Referring to FIG. 2B, once the semen layer 50, dense media layer 40 and horizontal interface 45 are provided within the vertical unit 30, the ICSI processing unit 1 of the invention is allowed to incubate, undisturbed, at room temperature, for a predefined amount of time to allow the desired, dense, motile sperm to swim down through the media layer 40 and contact the HA layer 20. In one or more embodiments, the incubation time may range from about 10 minutes to about 30 minutes. However, it has been found that incubation times less than 10 minutes and more than 30 minutes are also suitable for binding sperm to the HA layer 20 in accordance with the invention. As such, the processes of sperm swim-up, vertically oriented density selection and HA binding are combined into a single step, within a single vessel, for isolating fully developed motile sperm from undesired sperm that are under-developed and/or non-motile.

In accordance with swim-up, only those sperm that are motile will swim vertically from the semen layer 50 and through the dense media layer 40, while non-motile sperm are not able to cross the horizontal interface 45. Also not able to cross the horizontal interface 45 is non-motile matter including, but not limited to, dead sperm, dead cells, non-motile contaminating cells, cellular debris, soluble inhibitory factors, bacteria, contaminating non-sperm cells, and the like. However, in conventional swim-up processes the motile sperm may include underdeveloped motile sperm and physiologically immature sperm as well as motile contaminating bacteria.

The present invention prevents such motile underdeveloped motile sperm, physiologically immature motile sperm and motile contaminating bacteria from reaching the HA layer 20 by combining the swim-up process with the vertically oriented density selection step. In doing so, during the incubation period only motile sperm are able to swim down the vertical unit 30 through the dense media layer 40, which again is denser than the density of under-developed sperm, bacteria, cellular debris, non-sperm cells, seminal plasma, and the like. As such, the highly motile and fully formed sperm are allowed to traverse through the media layer 40 and contact the HA layer 20 for binding thereto. That is, the present invention simultaneously stops both non-motile matter and matter having densities less than the density of the underlying media (e.g., seminal plasma, under developed sperm and debris) at the horizontal interface 45, such that, this matter does not reach the HA layer 20.

Upon reaching the HA surface of the HA layer 20, only physiologically mature sperm 55 will have the required HA-receptor apparatus to bind to the HA layer 20. The physiological maturation of sperm requires the expression of an array of traits including the critical traits of DNA repair and proper meiosis as well as cytoplasm extrusion, re-packaging of DNA with protamines and remodeling of the sperm membranes to include receptors for binding HA. Development of these and other essential traits are all believed to be dependent upon a key chaperone protein that appears during two phases of sperm development. Chaperone proteins catalyze the proper folding of an array of different proteins, they stabilize the proteins during intracellular translocations and assist in membrane remodeling. In the absence of the chaperone, aberrant folding of the nacent polypeptides can occur, protein functionality can be lost during translocation and membrane remodeling can be impaired. Consequently, if the chaperone is deficient at critical moments in sperm development, a whole series of essential physiological developments may be compromised. Significant improvements in sperm quality, particularly reduction in aneuploidy and single-strand DNA breaks, strongly correlate to the trait of HA-binding.

Referring to FIGS. 2C and 2D, after the incubation interval, the spent semen layer 50 may be gently removed from over the media layer 40. This may be accomplished by carefully removing by pipette 70 essentially all of the spent semen layer 50, which now primarily contains contaminants.

Once the semen layer 50 has been removed, the ICSI processing unit 1 of the invention may be quickly and carefully inverted, preferably in one swift motion, to discard the dense media layer 40. As such, only the HA layer 20 having the desired physiologically mature sperm 55 with all the desired attributes obtained by the three selection techniques of the invention, i.e., swim-up selection, vertically oriented density selection and HA binding selection all performed simultaneously within a single vertically oriented device. Due to wetting principals, the surface of the HA binding layer may have a remainder of media film thereon in combination with the sperm 55 that is later rinsed off.

The vertical unit 30 is then detached from the support structure 10 as shown in FIG. 2E, either while the ICSI processing unit 1 is still inverted or after it is turned HA-side up again. In those embodiments wherein the vertical unit 30 is secured or affixed to the support structure 10 (e.g., attached by an adhesive), it is preferably done so with a connection that is easily and cleanly breakable for detachment of the vertical unit 30 from the support structure 10, without damaging the HA layer 20 containing bound sperm 55. For instance, the vertical unit 30 may simply be snapped off of the support structure leaving the HA-sperm bound layer intact. In other embodiments wherein the vertical unit 30 is simply placed on the support structure 10, during the step of inverting the unit 1 for draining media layer 40, the vertical unit 30 may be held in one hand while the support 10 in the other and inverted for drainage, followed by simply removing the vertical unit 30 from the ICSI processing unit 1.

In still other embodiments, the hollow vertical unit may be attached to the support structure using a mechanical, form fitting, non-leaking union, such that, the two parts fit tightly together by purely mechanical forces without leaking. The mechanical, form fitting, non-leaking union may be reversible and removable (e.g. removable by hand). For instance, the mechanical, form fitting, non-leaking union may include a rigid vertical unit 30 having an inside diameter that fits tightly over a round columnar form-fitting platform of essentially the same diameter as the inside diameter of the rigid vertical unit 30. As another example, the rigid vertical unit 30 may have a section of flexible plastic tubing of inside diameter slightly less than the outside diameter of a form-fitting columnar platform. This flexible tubing is stretched slightly to fit over and be held in place without leaking on the platform on the support structure.

Referring to FIG. 2F, the support structure 10 having the HA layer 20 containing bound sperm 55 is then turned HA-side up and rinsed with a sperm compatible fluid 90, such as, modified human tubal fluid (mHTF). This sperm compatible fluid 90 may be gently applied over the HA layer 20 and subsequently carefully removed therefrom by pipette, or alternatively, poured off. This step removes any residual density medium 40 without disturbing the bound sperm 55.

The surface of the HA layer 20 containing the bound sperm 55 may then be provided with a few additional drops of the sperm compatible fluid 90 to wet the sperm 55 containing HA surface as shown in FIG. 2G. The support structure 10 with the sperm 55 containing HA layer 20 may then be provided within an ICSI culture dish and flooded with mineral oil for ICSI sperm selection. In one or more embodiments, the support structure 10 may comprise an ICSI culture dish 10 whereby the HA layer 20 is provided on such ICSI culture dish 10, such that, no transfer of a support structure to a culture dish is necessary since the HA layer 20 is already within the culture dish. It should also be appreciated that the support structure, whether it is a structure that is to be transferred to a culture dish or is the culture dish itself, may be provided with more than one HA layer 20 and vertical unit 30 for processing multiple samples at the same time. These samples may be identical samples or different samples.

In accordance with the various embodiments of the invention, a large field of well separated, bound, motile, physiologically mature sperm 55 are provided in a clean support medium, essentially free of non-motile sperm, dead or contaminating cells, inhibitory plasma, bacteria and residual density medium. The ICSI operator may then use a microscope stage and micropipette to select bound sperm 55 for subsequent known ICSI processing techniques. That is, the ICSI units of the invention may be placed on a stage of an inverted microscope to provide ready access to the micropipettes used to recover and manipulate sperm and oocytes for the practice of ICSI.

The time required for the complete sperm processing in accordance with the present invention, from setting-up the device to the moment at which sperm are available for selection, is typically less than about 30 minutes. Once bound to the HA layer, the sperm are stable and separated from the semen sample. Alternatively, the operator may choose to allow the ICSI processing unit 1 to continue to incubate for extended periods while retaining sperm stability and functionality. The sperm preparation steps may also be completed and the bound sperm retained under oil in the ICSI culture dish until wanted. As such, the present invention provides convenience and efficiency to ICSI processing.

The present methods, systems and apparatus for ICSI processing in accordance with the various embodiments of the invention provide the operator with an enhanced measure of control over the selection and recovery process. The binding of sperm to the HA layer can be observed during sperm swim down into the density layer by placing the ICSI processing unit 1 on an inverted microscope and observing the HA layer. In so doing, the operator is able to determine whether or not a sufficient number of bound sperm have arrived at the HA layer to obtain a fruitful recovery of bound sperm. If the operator finds that the bound sperm are too crowded for easy selection of individual bound sperm, some of the bound sperm may be washed off the HA layer by repeated rinsing of the layer after removal of the vertical unit.

While not meant to limit the invention in any manner, a number of examples are provided below in connection with the present invention. As is shown in these examples, the actual rate at which sperm bind to the HA layer is a function of four factors: the percentage of motile, HA-binding sperm in the semen sample (the HBA® Sperm-Hyaluronan Binding Assay score); the concentration of sperm in the semen sample; the distance the sperm must swim to reach the HA layer, i.e., the volume of density media intervening between the HA layer and the semen; and the incubation time. The first two are easily measured and the latter two are under the direct control of the operator. It has been found that under typical conditions where the semen has at least a million motile HA-binding sperm per mL and the separation distance is about 7 mm, a suitable population is bound to the HA layer in less than ten minutes.

### EXAMPLES

Examples 1 and 2 refer to methods of fabricating ICSI processing units 1 in accordance with one or more embodiments of the invention, as well as the resultant units 1 made by such methods.

### Example 1

An ICSI processing unit 1 was fabricated using a glass microscope cover slip that was approximately 0.5 mm thick and 2.5 cm×2.5 cm (e.g., a no. 4 cover slip). The glass microscope cover slip was coated with hyaluronan (HA) as follows: A base coat containing acrylic polymer Hydak G-23 from Biocoat, Inc., in acetone, was amended with a multifunctional isocyanate cross linking agent, such as, hexamethylene diisocyanate, and allowed to dry at 60° C. for ten minutes. An aqueous top coat containing 0.30% sodium hyaluronate and 0.10% Triton X-100 surfactant was applied to the dried base coat, water was allowed to evaporate and the coated cover slip was cured at 60° C. for 16 hours. Following curing the coated cover slip was immersed in deionized water for 30 minutes, rinsed and dried. It should be appreciated that a variety of alternative solvents, acrylic polymers, surfactants and crosslinking agents may be substituted to produce an equivalent coating of hyaluronan.

A hollow tubular chamber 3 cm in length was cut off the base segment of a 1 mL polypropylene disposable pipette tip, and glued to the hyaluronan-coated cover glass using Weldwood® Contact Cement. Loctite® Sumo Glue and Scotch® Quick Dry Adhesive were also used as the glue in other fabrications of this unit 1. Since some of the hollow tubular chambers were fabricated from a tapered disposable pipette tip, some of the hollow tubular chambers were also slightly tapered and had an internal diameter of approximately 0.80 cm. After drying, each of these chambers held a column of aqueous fluid over 3 cm tall without leaking.

### Example 2

An alternate ICSI processing unit 1 of the invention includes a small area of a Becton Dickinson Falcon 1006 polystyrene culture dish coated with a layer of hyaluronan. The coating was a single coat consisting of 0.40% sodium hyaluronate plus 0.05% of a non-ionic surfactant 1, plus a very small amount, approximately 0.002%, of a crosslinking agent. Suitable crosslinking agents include, but are not limited to, a polyfunctional epoxide, 1,2,7,8-diepoxyoctane, or a polyfunctional aziridine, 1-aziridinepropanoicacid, 2-methyl-,1,1′-[2-ethyl-2-[[3-(2-methyl-1-aziridinyl)-1-oxopropoxy]methyl]-1,3-propanediyl]ester. After curing at 60° C. for four hours, the coated culture dish was immersed in DI water, rinsed and dried.

The hollow vertical unit (also referred to herein as “column”) comprised a borosilicate glass tube, 1.0 cm internal diameter and 3 cm long. The glass tube was heated to 550° C. on a thermostatted hot plate and then one end of it was pushed with moderate pressure into the base of the polystyrene culture dish, at the location corresponding to the hyaluronan-coated area, and allowed to cool. In this manner, the tube was sealed to the culture dish by local melting of the plastic, forming a column open at the top and sealed entirely shut at the bottom so as to prevent fluid leakage. The column held aqueous fluid for hours without leaking.

In this embodiment, the vertical unit is part of the ICSI culture dish. It should be appreciated that many other heat-stable, including aluminum or quartz tubing, may be used to form the hollow vertical unit of the invention. Similarly, plastic hollow vertical units may be attached to plastic bases with the use of compatible glues.

Examples 3-9 refer to methods of utilizing the various ICSI processing units 1 fabricated in accordance with the embodiments of the invention.

### Example 3

Freshly collected human semen was obtained by masturbation and incubated at room temperature for 30 minutes to liquefy. A portion of the semen was mixed with an equal volume of Human Tubal Fluid (HTF of Irvine Scientific) supplemented with 5 mg/mL of serum albumin (HTF+SA) and mixed by gentle agitation to obtain a diluted semen sample. In general, a suitable range for mixing HTF+SA with the semen varies from one part HTF+SA plus one part semen to nine parts HTF-FSA plus one part semen. The diluted semen sample that was used contained approximately 25 million motile sperm per mL.

An ICSI processing unit, as described in Example 1, was implemented by adding 0.80 mL of Nidacon PureSperm® 80 gradient medium to the bottom of the ICSI unit's column (i.e., vertical unit), wetting the hyaluronan layer. An aliquot of 0.20 mL of the diluted raw semen was carefully layered on top of the gradient medium and the unit 1 incubated at room temperature (18-30° C.) without agitation for 20 minutes. During the incubation, the unit 1 may be placed on an inverted microscope for viewing the hyaluronan layer at 100 to 400× magnification. Within 5 to 10 minutes, a population of sperm were bound to the hyaluronan layer. These bound sperm were clearly identified by their vigorously beating tails with no progressive movement of the sperm itself.

The unit 1 containing the sperm layer, media layer and HA layer may be allowed to stand and incubate for a time period of about 10 to about 60 minutes, followed by harvesting the bound sperm. First, the majority of the aqueous material was removed by pipette, collecting the material from the top surface of the liquid, working down, and moving the pipette tip around the inside edge of the column. In doing so, spent raw semen was collected without contaminating the underlying liquid column. When about 3 mm of liquid remained undisturbed covering the hyaluronan layer, the ICSI processing unit 1 was rapidly inverted in a single motion and the remaining liquid contents flowed down and out of the open end of the column. With the ICSI processing unit in an inverted position, the column was snapped off the base (i.e., support substrate) and discarded leaving the hyaluronan layer with a thin film of aqueous covering the bound sperm.

The base was returned to an upright position and the hyaluronan layer was gently rinsed using a drop of HTF+SA which was expressed at the end of a pipette and carefully lowered until it contacted the hyaluronan layer. This liquid was then slowly removed by pipette whose tip was placed at the edge of the hyaluronan coating. This rinsing was repeated once more and then two drops of HTF+SA were applied to the hyaluronan layer and the device was placed into a sterile culture dish prepared for ICSI. Additional drops of aqueous material required for ICSI processing, such as drops of polyvinylpryrrolidone (PVP) or drops to contain the oocyte, were added to the culture dish according to the usual ICSI procedure. The culture dish, including the additional drops and the developed device, were flooded with sterile mineral oil and ICSI was performed. In ICSI, sperm were selected from among those bound to the hyaluronan layer, recovered by gentle aspiration into the micropipette, washed and stunned in a droplet of PVP and used for injection into the oocyte.

### Example 4

Like that of Example 3, human semen was freshly collected and diluted, and then provided within a ICSI processing unit of the invention as described in Example 2. The processing in this example continued as described in Example 3, whereby the sample was inoculated and developed, however, in this example the developed, rinsed unit 1 did not have to be placed into a culture dish since the device of Example 2 comprises a part of the ICSI culture dish. After rinsing and adding the final two drops of HTF-FSA, the rest of the dish was loaded with the additional ICSI drops, the dish was flooded with mineral oil, and ICSI was performed.

### Example 5

The following example includes a subset of examples showing the influence of different volumes of density medium layer 40 has on sperm swimming therethrough. In order to reach the HA layer, motile mature sperm must pass through the density medium swimming randomly. The length of the path that sperm directed to the HA layer must follow is defined by the height of the liquid column that they must swim through, which in turn, is defined by the dimensions of the hollow vertical unit and the volume of density medium provided therein.

Three different ICS units as in Example 2 (units 5-A, 5-B, 5-C) were loaded with different volumes of NidaCon PureSperm® 80 density mediums, respectively, 0.30 mL, 0.80 mL and 1.4 mL. Once each unit was loaded with sperm, containing approximately one million motile sperm, a timer was started and sperm binding to the HA layer was observed in the microscope during processing. The results showed that the unit 5-A containing the least volume of dense media, i.e., the unit containing 0.30 mL, developed a dense population of bound sperm in less than five minutes. The unit 5-B containing the middle volume of dense media, i.e., the unit containing 0.80 mL, required about 10 to 15 minutes to achieve a similar density of bound sperm, while the unit 5-C the largest volume of dense media, i.e., the unit containing 1.4 mL, required more than 30 minutes to achieve a similar density of bound sperm.

When smaller diameter vertical units 30 were used, smaller volumes of density medium were required to create liquid columns similar in height to those described above. The approximate rates at which sperm bound to the HA layer were similar for devices with essentially identical heights of liquid. When all else was held constant, the height of the liquid intervening between the HA layer and the sperm sample was the dominant determinant of how fast the HA layer was populated with bound sperm. In one or more embodiments, ICSI units 1 of the invention having a hollow vertical unit 30 of about 0.8-1.0 cm internal diameter, a volume of density medium layer 40 of about 0.75-0.80 mL, corresponding to a column height of about 6.5 mm to 7 mm, it has been found that sperm bind to the HA layer within about 5 to about 30 minutes. Accordingly, the desired concentration of sperm bound to the HA layer is not only dependent upon the concentration of sperm within the sperm sample, but also on the dimensions of the vertical unit and the volume of the density medium layer provided therein.

### Example 6

The following example includes another subset of examples showing the affects that different density medium layers have on sperm swimming therethrough.

First, four different ICSI units (units 6-A, 6-B, 6-C, 6-D), as in Examples 1 and 2, were loaded, as described in Example 3, with 0.75 mL of four different density media layers (i.e., of different chemical compositions) each at pH 7.4 and adjusted to a density ranging from about 1.09 g/mL to about 1.11 g/mL. These different density media were Pure Sperm®80 of Nidacon, Isolate® of Irvine Scientific, Percoll® of Sigma, and OptiPrep® of Sigma. Each unit was inoculated with 0.20 mL of 1:2 diluted raw semen, incubated for 20 minutes, developed and observed. Observations showed that small differences existed in the concentration of sperm bound to the HA layer. That is, each of the foregoing dense media having densities ranging from about 1.09 g/mL to about 1.11 g/mL was effective as a density barrier for sperm selection in accordance with the invention.

A fifth ICS unit (unit 6-E) was loaded with 0.75 mL of OptiPrep having a density adjusted up to 1.265 g/mL. In this example that has a higher density, it was found that essentially no motile sperm bound to the HA layer. Again, in accordance with one or more embodiments of the invention, it is preferred that the dense media layer 40 has a density ranging from about 1.050 g/mL to about 1.150 g/mL.

### Example 7

The following example includes still another subset of examples showing the affects of using compromised and/or frozen sperm samples in the present ICSI units of the invention as described in Example 2 and processed as in Example 3.

A first unit (unit 7-A) was provided with 0.20 mL of an oligospermic semen sample (i.e., a compromised sperm sample) having an overall sperm concentration 5 million/mL. It took about 10 to 30 minutes longer for the compromised sperm to bind to the HA layer (as compared to the sperm of Example 3). It is believed that the longer time was due to a lower concentration of sperm in the inoculum plus a lower proportion of hyaluronan (mature) sperm in the inoculum.

A second unit (unit 7-B) was provided with a freshly thawed sample of cryopreserved sperm residing within an egg yolk preservation medium. This sample was diluted with an equal volume of HTF+SA and developed as in Example 3. It was found that while sperm did in fact bind to the HA layer, it took longer to do so as compared to the sperm of Example 3, perhaps due to the slower average swimming speed of the freshly thawed semen in combination with the viscosity of the preservation medium. A third unit (unit 7-C) was provided with a freshly thawed sample of cryopreserved sperm residing within a preservation medium that did not contain egg yolk. While binding did occur, this sample also developed more slowly than the fresh semen sample of Example 3.

Regardless of the speed of sperm-HA binding, it was found that these compromised and/or frozen sperm samples were suitable for use in the present invention to provide a sufficient quantity of sperm bound to the HA layer that were free from the contaminants residing within the inoculum and the compromised/frozen sperm sample itself.

### Example 8

The following example includes a subset of examples showing the affects of temperature on the present invention.

Since ICSI is best performed at body temperature (approximately 37° C.) to ensure stability of the oocyte, units prepared as in Example 2 were developed as in Example 3 but at 37° C. and compared to a duplicate device developed at room temperature (25° C.). Fewer sperm were found bound to the 37° C. unit than in the comparable unit developed at room temperature. Sperm swim more vigorously at 37° C., than at temperatures below 31° C., such that, this more vigorous swimming may result in a break of the sperm-HA bond, thereby freeing the sperm.

In one or more embodiments, it is preferred that the units be processed at room temperature where sperm-HA binding is increased over that of higher temperatures (e.g., those above 31° C.). Other embodiments may be operated at temperatures above 31° C., such as for example at 37° C., where sperm-HA binding still occurs, just not as prevalent as that of operating temperatures below 31° C.

### Example 9

The following example includes a subset of examples showing the affects of viscosity of the semen sample on the present invention.

Some semen samples fail to effectively liquefy tending to trap and retain motile sperm in the semen gel. To liquefy the sample, it is treated with proteolytic enzymes. In this example, a semen sample that failed to liquefy in 30 minutes at room temperature was mixed with an equal volume of HTF+SA that also contained 100 Unit/mL of α-chymotrypsin from bovine pancreas. The mixture was immediately put onto a unit prepared as in Example 4 and incubated for 30 minutes. The developed unit yielded a sufficient quantity of sperm bound to the HA layer.

When the HTF+SA surrounding the bound sperm in the finished, developed device was assayed for chymotrypsin activity with benzoyl-L-tyrosine ethyl ester at pH 7.8, less than 0.01 Unit/mL was found. As such, it has been found that the present invention separates sperm from the α-chymotrypsin as well as immature sperm and contaminating water-soluble inhibitory factors. It has also been found that the invention is suitable for use with highly viscous semen samples as described above.

Accordingly, the various embodiments of the invention remove undesirable sperm retained by one principal of separation by applying one or more additional ICSI processing techniques simultaneously within a single processing unit, thereby improving sperm quality for subsequent ICSI processing. The embodiments of the invention provide an easy, quick, and efficient approach to yielding sperm in a simple, directly accessible form, ready for ICSI selection, as compared to the prior art approaches which are tedious, labor intensive, time-consuming, and also do not yield sperm in a simple, directly accessible manner for ICSI selection.

While the present invention has been particularly described, in conjunction with a specific preferred embodiment, it is evident that many alternatives, modifications and variations will be apparent to those skilled in the art in light of the foregoing description. It is therefore contemplated that the appended claims will embrace any such alternatives, modifications and variations as falling within the true scope and spirit of the present invention.

Thus, having described the invention, what is claimed is:

1. A method for preparing sperm for Intracytoplasmic Sperm Injection (ICSI) comprising:
providing a processing unit comprising a hollow vertical unit attached to a horizontal support structure, said hollow vertical unit having a first end adjacent said support structure to provide a leak-proof seal and a second end having an opening, a hyaluronan layer residing on said horizontal support structure such that at least a portion of said hyaluronan layer is exposed within said hollow vertical unit;
depositing a density gradient media layer into said opening of said hollow vertical unit to entirely cover said exposed hyaluronan layer within said hollow vertical unit;
depositing a semen layer directly over said density gradient media layer to form a defined horizontal density gradient interface between said density gradient media layer and said semen layer, said density gradient media layer having a density greater than a density of said semen layer and lower than a density of fully-formed motile sperm;
allowing said processing unit containing said semen layer, said density gradient media layer and said hyaluronan layer to stand undisturbed for an amount of time to allow fully-formed motile sperm swim in a downward vertical direction from said semen layer, through said defined horizontal density gradient interface, through said density gradient media layer and toward said hyaluronan layer to bind to said hyaluronan layer residing within said hollow vertical unit;
removing said semen layer and said density gradient media layer; and
accessing said hyaluronan layer having said fully-formed motile sperm bound thereto by removing said hollow vertical unit from said horizontal support structure.

2. The method of claim 1 wherein said support structure comprises a transparent material for viewing said hyaluronan layer to observe binding of said fully-formed motile sperm to said hyaluronan layer.

3. The method of claim 1 wherein said density gradient media layer has a density ranging from about 1.050 g/mL to about 1.150 g/mL.

4. The method of claim 1 wherein said density gradient media layer has a pH ranging from about 7.2 to about 7.6.

5. The method of claim 1 wherein said density gradient media layer is selected from the group consisting of PVP-coated silica media, silane-coated silica media and iodixanol media.

6. The method of claim 1 wherein said semen sample is selected from the group consisting of neat semen, diluted raw semen, freshly thawed frozen semen, treated semen, compromised semen, and combinations thereof.

7. The method of claim 1 wherein said fully-formed motile sperm bind to said hyaluronan layer without the need for centrifugal forces.

8. The method of claim 1 wherein said amount of time that said processing unit is allowed to stand ranges from about 5 minutes to about 30 minutes.

9. The method of claim 1 wherein said defined horizontal density gradient interface and said density gradient media layer prevent matter from contacting and binding to said hyaluronan layer, said matter selected from the group consisting of motile underdeveloped motile sperm, motile physiologically immature motile sperm, motile contaminants, non-motile sperm, under-developed sperm, dead sperm, dead cells, bacteria, cellular debris, non-sperm cells, seminal plasma, non-motile contaminating cells, soluble inhibitory factors, and combinations thereof.

10. The method of claim 1 further including rinsing said hyaluronan layer having said fully-formed motile sperm bound thereto prior to ICSI sperm selection.

11. The method of claim 10 further including providing said support structure having said hyaluronan layer with said fully-formed motile sperm bound thereto within an ICSI culture dish for ICSI sperm selection.

12. The method of claim 1 wherein said support structure comprises an ICSI culture dish for ICSI sperm selection.

13. An apparatus for preparing sperm for Intracytoplasmic Sperm Injection comprising:
a support structure having a horizontal plane;
a hyaluronan layer on said support structure;
a hollow vertical unit on said support structure having a length that extends vertically with respect to said horizontal plane of said support structure, said hollow vertical unit having a first end adjacent said support structure to provide a leak-proof seal and having a second end space apart from said support structure, said second end having an opening for entry of a fluid column into said hollow vertical unit, said hyaluronan layer residing at said first end of said hollow vertical unit and having a surface area of hyaluronan layer exposed within said hollow vertical unit.

14. The apparatus of claim 13 wherein said support structure comprises a transparent material for viewing said hyaluronan layer.

15. The apparatus of claim 13 wherein said hyaluronan layer resides on said support structure, such that, said hollow vertical unit contacts said hyaluronan layer on said support structure to form said leak-proof seal between said hollow vertical unit and said hyaluronan layer.

16. The apparatus of claim 13 wherein said hyaluronan layer comprises a region of hyaluronan residing at a specific location on said support structure, whereby said hollow vertical unit contacts said support structure to form said leak-proof seal between said hollow vertical unit and said support structure, whereby said region of hyaluronan resides entirely within said hollow vertical unit, such that, said hollow vertical unit encases said region of hyaluronan.

17. The apparatus of claim 16 wherein said hollow vertical unit is removably attached to said support structure.

18. The apparatus of claim 17 wherein said hollow vertical unit is securely connected to said support structure using a breakable connection or a mechanically removable connection.

19. The apparatus of claim 16 wherein said breakable connection comprises a breakable chemical connection or a breakable thermally-bonded connection, a user being able to physically break said breakable connection to remove said hollow vertical unit from said support structure and gain access to said hyaluronan layer.

20. A system for preparing sperm for Intracytoplasmic Sperm Injection comprising:
a support structure having a horizontal plane;
a hyaluronan layer on said support structure;
a hollow vertical unit in communication with said support structure having a length that extends vertically with respect to said horizontal plane of said support structure, said hollow vertical unit having a first end adjacent said support structure to provide a leak-proof seal and having a second end space apart from said support structure, said second end having an opening for entry of a fluid column into said hollow vertical unit, said hyaluronan layer residing at said first end of said hollow vertical unit and having a surface area of hyaluronan layer exposed within said hollow vertical unit;
a density gradient media layer provided within said hollow vertical unit to entirely cover said exposed hyaluronan layer within said hollow vertical unit;
a semen layer directly over said density gradient media layer to form a defined horizontal density gradient interface therebetween,
wherein said density gradient media layer has a density greater than a density of said semen layer and lower than a density of fully-formed motile sperm, whereby after standing undisturbed for an amount of time fully-formed motile sperm swim vertically downward from said semen layer, through said defined horizontal density gradient interface, through said density gradient media layer and toward said hyaluronan layer to bind thereto, while non-motile matter, motile underdeveloped motile sperm, motile physiologically immature motile sperm, and motile contaminants are prevented from contacting said hyaluronan layer by said defined horizontal density gradient interface and said density gradient media layer.
